SEQUENCE LISTING (21 sequences)

SEQ ID NO: 1 - protein, 123 aa, Lama glama
QVQLQDSGGGLVQAGDRLSLSCAASGRTFSSYDMAWFRQAPGKEREFVAAITTSEGTWYGDAGKGRFTIARVNAKNTVYLHMNRLKPEDTAVYYCAASNQGGSLQISTNYNYWGQGTQVTVSS

SEQ ID NO: 2 - protein, 123 aa, Lama glama
QVQLQESGGGLVQAGDSLRLSCAASGRTFSSYAMGWFRQAPGKEREFVAAVSSSNSPWYGDSIKGRSTIHRDNAKNTVYLYMNNLKPEDTAVYYCAADLEGERLGESSEYDYWGQGTQVTVSS

SEQ ID NO: 3 - protein, 127 aa, Lama glama
QVQLQESGGGLVQPGGSLRLSCAASGFTFKYYAMSWVRQAPGKGLEWVSYINDNGGYTDYSDSVKGRFTISRDNAKNTLYLHMNRLKPEDTAVYFCAKWDTDAVSSSRYKTHNGDIRGPGTQVTVSS

SEQ ID NO: 4 - protein, 123 aa, Lama glama
QVQLQESGGGLVQPGGSLRLSCAASGRTFSSYAMGWFRQAPGKERQFVAAVSSSDSPWYGDSMKGRSTIHRDNAKNTVYLYMNNLKPEDTAVYYCAADLEGERLGESSEYDYWGQGTQVTVSS

SEQ ID NO: 5 - protein, 125 aa, Lama glama
QVQLQESGGGLVQAGDSLRLSCAASGGALYDMAWFRQAPGKEREFVAVISYDGSATSYRDSVKGRFTISRENAKNMARNMAYLQMNALKPEDTAVYYCAAKGPTGTLHTSGYRIWGQGTQVTVSS

SEQ ID NO: 6 - protein, 125 aa, Lama glama; identical to SEQ ID NO: 5

SEQ ID NO: 7 - protein, 126 aa, Lama glama
QVQLQESGGGLVRPGGSLRLTCATSGFSLDYYPIAWFRQAPGKEREGLSCISSSDGGAYYGDAVKGRFTMSRDSAENTVYLHMNSLKPEDTAVYYCATSKRYWRNCDVTDYDYHYWGQGTQVTVSS

SEQ ID NO: 8 - protein, 126 aa, Lama glama
QVQLQESGGGLVQPGGSLRLSCATSGFSLDYYPIAWFRQAPGKEREGLSCVSSSDGGTYYGDAAKGRFTMSRDSAENTVTLHMNSLKPEDTAVYYCATSKRYWRNCDVTDYDYHYWGQGTQVTVSS

SEQ ID NO: 9 - protein, 126 aa, Lama glama
QVQLQESGGGLVQAGDSLTLSCAVSGGTLSMFSMGWFRQAPGLEREFVAIINDRESIRRYQDSVKGRFTISKDNAKNLVYLQMNNLKPVDTAVYYCAADTTYPAVAPTSVGQYDYWGQGTQVTVSS

SEQ ID NO: 10 - protein, 125 aa, Lama glama
QVQVQKSEGGLVQPGGSLRLSCAVSGRTDSISAMGWFRQAPGKEREFVVAIRWIEGGTWYGDPAAGRFTIARDIAKNTGYLQMNRLNPDDTAVYFCAAAKFGPSKLQFSQGYDYWGQGTQVTVSS

SEQ ID NO: 11 - protein, 118 aa, Lama glama
QVKLAGTLGGLVQLGDSLQLSCVASGRTDVMGWFRQAPGKEREFAAAITGSDGTFYGDAGRGRFTIARDNTKNTVYLNLNRLKPEDTAVYYCAADGSPLQIASTYKYWGQGTQVTVSS

SEQ ID NO: 12 - protein, 124 aa, Lama glama
QVQLQDSGGGLVQAGTSLRLSCVAKGGLYGLGWFRQAPGKEREFVAAISGSGGSTYYGDSVRGRFTISRDNTKNTMYLQMNSLKPEDTAIYFCAARTQYSASDYWWQQGEYGYWGQGTQVTVSS

SEQ ID NO: 13 - protein, 127 aa, Lama glama
QVQLQESGGGLVQPGGSLRLSCEASGFTFSYYAMSWVRQAPGKGLEWVSYINSDGGYTSYADSVKGRFIISRDNAKNILYLHMNSLKPEDTAVYFCAKWRTDAVSSSRYKTNDYEYRGPGTQVTVSS

SEQ ID NO: 14 - protein, 125 aa, Lama glama
QVQLQKSGGGLVQAGGSLRLSCARGSGTFSPYAMGWFRQAPGEERQFVAAITWSSDSPWYGDSAKGRCTIARDITKNTVYLNIFGLKPEDTAVYYCAESSGATYGLALPSAYEHWGRGTQVTASS

SEQ ID NO: 15 - protein, 127 aa, Lama glama
QVQGAGLWGGLVQLGGSLRLSCAASGFTFGYYAMSWVRQAPGKGLEWVSYINDNGGYTAYTDPVKGRFTISRDNAKNILYLHMNNLKPEDTAVYFCAKWGTDAVSSSRYKSHDYDSRGPGTQVTVSS

SEQ ID NO: 16 - protein, 125 aa, Lama glama
QVQLQESGEGLVQAGASLKLSCEASLGGLYGMGWFRQAPGKEREFVAAISGSGGTTYYEDSVRGRFTISRDNAKSTVFLQMNNLKPEDTAVYYCAARTSYSASNAWNQLREYGYWGQGTQVTVSS

SEQ ID NO: 17 - protein, 122 aa, Lama glama
QVQLQESGGGLVQAGGSLRLSCAGVGRTISPYNMGWFRQALGKERDFVAAIRWSGSDTNYADSVKGRFTISRDNAENTVYLQMRNLNPEDTAVYYCAAGGDVFGSALTYHHWGQGTQVTVSS

SEQ ID NO: 18 - protein, 119 aa, Lama glama
QVQLQESGGGLVQPGGSLRLSCAASGSIRSINAMGWYRQAPGKQREFVARITTGGTIGYADSVKGRFTISRDNAKNTMYLQMNSLKPEDTAVYYCNADIGTWSSPYYYWGQGTQVTVSS

SEQ ID NO: 19 - protein, 122 aa, Lama glama
QVQLQDSGGGLVQAGGSLRLSCAGVGRTISPYNMGWFRQALGKERDFVAAIRWSGSDTNYADSVKGRFTISRDNARNTVYLQMNNLNPEDTAVYYCAAGGDVFGSALTYHHWGQGTQVTVSS

SEQ ID NO: 20 - protein, 119 aa, Lama glama
QVQLQESGGGLVQPGGSLRLSCAASGSIRSINVMGWYRQAPGKQRELVALITTGGSPSYVDSVKGRFTISRDNAKSTIYLQMDSLKPEDTAVYYCNADIGTWSSPFYYWGQGTQVTVSS

SEQ ID NO: 21 - protein, 122 aa, Lama glama
QVQLQESGGGLVQAGGSLRLSCEGVGRSISPYNMGWFRQAPGKERDFVAAIRWSGSDTNYADSVKGRFTISRDNARNTVYLHMNSLNPEDTAVYYCAAGGDVSGSALSYHYWGQGTQVTVSS

(19) United States
(12) Patent Application Publication
(10) Pub. No.: US 2011/0123546 A1
(43) Pub. Date: May 26, 2011

(54) Compositions Comprising Antibodies or Antibody Fragments

(76) Inventors: Leo Gerardus J. Frenken, Vlaardingen (NL); Lars-Göran Lennart Hammarström, Huddinge (SE); Neha Pant, Huddinge (SE)

(21) Appl. No.: 12/936,841

(22) PCT Filed: Apr. 1, 2009
(86) PCT No.: PCT/EP09/53880

§ 371 Date: Dec. 21, 2010

(30) Foreign Application Priority Data
Apr. 18, 2008 (EP) 08154794.5

(51) Int. Cl. A61K 39/42; A61P 31/14

(52) U.S. Cl. 424/159.1

(57) ABSTRACT

The present invention relates to food products or pharmaceutical preparations comprising a synergistic combination of at least two different antibodies or antibody fragments which are directed against a virus, preferably against rotavirus.

### FIELD OF THE INVENTION

The present invention relates to compositions comprising antibodies or antibody fragments. More in particular, it relates to food products or pharmaceutical preparations comprising antibodies or antibody fragments which are directed against a virus. The invention also relates to a method for preparing food products and pharmaceutical preparations comprising the antibodies or antibody fragments and the use of these products to deliver health benefits to humans.

### BACKGROUND OF THE INVENTION

Antibodies are protein molecules belonging to a group of immunoglobulins generated by the immune system in response to an antigen. When antigens invade humans or animals, an immunological response is triggered which involves the production of antibodies by B-lymphocytes. By this immunological response amongst others, micro-organisms, larger parasites, viruses and bacterial toxins can be rendered harmless. The unique ability of antibodies to specifically recognise and bind with high affinity to virtually any type of antigen, makes them for a variety of applications.

The structure of most antibody molecules is based on a unit comprising four polypeptides, two identical heavy chains and two identical light chains, which are covalently linked together by disulphide bonds. Each of these chains is folded into discrete domains. The C-terminal regions of both heavy and light chains are conserved in sequence and are called the constant regions, comprising one or more so-called C-domains. The N-terminal regions of the heavy and light chains, also known as V-domains, are variable in sequence and determine the specificity of the antibody for its antigen. The regions in the variable domains of the light and heavy chains (V_{L} and V_{H}, respectively) responsible for antigen binding activity are known as the hypervariable or complementarity determining regions (CDR). The combined V_{L} and V_{H} domain is also known as the antigen binding site of an antibody. Each antibody molecule comprises two antigen binding sites and is therefor bi-valent.

WO 94/04678 discloses immunoglobulins capable of exhibiting the functional properties of the four-chain immunoglobulins described above, but which comprise two heavy polypeptide chains and which furthermore are devoid of light polypeptide chains. Fragments corresponding to isolated V_{H} domains (hereinafter VHH) are also disclosed. WO 94/25591 (Unilever) discloses methods for the preparation of such-antibodies or fragments thereof on a large scale comprising transforming a mould or yeast with an expressible DNA sequence encoding the antibody or fragments. The immunoglobulins described in WO 94/04678, which may be isolated from the serum of Camelids, do not rely upon the association of heavy and light chain variable domains for the formation of the antigen-binding site but instead the variable domain of the heavy polypeptide chains alone naturally form the complete antigen binding site.

The VHH binding domains of these immunoglobulins, hereinafter referred to as “heavy-chain immunoglobulins”, are thus quite distinct from the V_{H} domain obtained from the heavy chains of common (four-chain) immunoglobulins (by degradation or direct cloning) which contribute only in part to the antigen-binding site and require a light chain V_{L} partner for antigen-binding, thus forming a complete antigen binding site. The combined V_{L} and V_{H} domain are also known as Fv fragment.

Antibodies or fragments thereof, have found application in a variety of fields where the specific nature of the antibody-antigen interaction can be used to advantage. These include diagnosis, therapy, immunoassays and purification processes. The use of antibodies, or fragments thereof, in inhibiting viral infection has received attention, for instance during passive immunisation by the administration of neutralising antibodies.

WO 99/23221 discloses multivalent antigen binding proteins for inactivating phages. The hosts may be lactic acid bacteria which are used to produce antibody binding fragments which are recovered.

WO 00/65057 is directed to the inhibition of viral infection, using monovalent antigen-binding proteins. The antigen-binding protein may be a heavy chain variable domain derived from an immunoglobulin naturally devoid of light chains, such as those derived from Camelids as described in WO 94/04678. WO 00/65057 discloses transforming a host with a gene encoding the monovalent antigen-binding proteins. Suitable hosts can include lactic acid bacteria. This disclosure relates to the field of fermentation processing and the problem of phage infection which hampers fermentation.

Specifically, llama VHH fragments are used to solve the problem of bacteriophage infection by neutralising Lactoccoccus lactis bacteriophage P2.

Both WO 00/65057 and WO 99/23221 involve the use of antibody fragments harvested from a microbial expression system.

WO-A-2006/056306 (Unilever) discloses a delivery system for delivering antibodies to the gastro-intestinal tract, comprising heavy chain immunoglobulins of the VHH or VNAR type or domain antibodies of the heavy or light chains of immunoglobulins or fragments thereof, wherein the immunoglobulins or fragments thereof are active in the gut.

WO-A-2007/019901 (Unilever) discloses food products and pharmaceutical preparations comprising antibodies or antibody fragments which are active in the gut, in combination with probiotic micro-organisms.

It is important to ensure that the antibody or antibody fragments are active in a specific region of the body, for example the stomach and/or the gut. Alternatively, the antibody or antibody fragments may be active in the process of the food preparation.

Moreover, antibody or antibody fragments are expensive, which restricts their application in specific countries in the developing and emerging markets, where they are especially needed to combat viral infections.

Thus, here is a constant need for alternative or improved food products or pharmaceutical preparations comprising an antibody or antibody fragment which are directed against a virus.

The present inventors have surprisingly found that this object can be achieved by the food products or pharmaceutical preparations according to the present invention, comprising a synergistic combination of at least two different antibodies or antibody fragments which are directed against a virus.

### SUMMARY OF THE INVENTION

According to a first aspect of the invention, there is provided a food product or pharmaceutical preparation comprising a synergistic combination of at least two different antibodies or antibody fragments which are directed against a virus.

According to a second aspect of the invention, there is provided a method for making a food product or pharmaceutical preparation according to the first aspect, comprising adding the antibodies or antibody fragments during the manufacture of the food product or pharmaceutical preparation or an ingredient thereof.

According to a third aspect of the invention, there is provided the use of the food product or pharmaceutical preparation according to the first aspect of the invention or made according to the second aspect of the invention to combat enteropathogenic micro-organisms, in particular viral infections. This aspect of the invention provides the food product or pharmaceutical preparation according to the first aspect of the invention or made according to the second aspect of the invention for use as medicament, in particular for use to combat enteropathogenic micro-organisms, in particular viral infections.

According to a fourth aspect of the invention, there is provided a dispensing implement for use with a food product wherein the dispensing implement is coated on at least one surface with antibodies or anti-body fragments and/or at least one micro-organism producing at least one of the antibodies or fragments.

### DETAILED DESCRIPTION OF THE INVENTION

The present invention relates to a food product or pharmaceutical preparation. The term “food product” as used herein means food products in the widest sense which are suitable for humans and animals. It also encompasses beverages and powders that have to be dissolved in order to prepare a beverage (powder drinks). Alternatively the food product can be a product which can be mixed in or sprinkled on other food products, or can be a syrup to be mixed with other drinks. In another embodiment it can be a food product like porridge.

As a first element, the food product or pharmaceutical preparation of the present invention comprises a synergistic combination of at least two different antibodies or antibody fragments which are directed against a virus. The virus is preferably a food-born virus such as rotavirus. In another preferred embodiment, the virus is an enteropathogenic virus such as rotavirus. The antibodies or antibody fragments are preferably active in the gut, but they can also be effective if they are only active during the food preparation.

There are various possibilities in which the food product or pharmaceutical preparation can comprise the synergistic combination of at least two different antibodies or antibody fragments which are directed against a virus. First, it may comprise two or more separate antibodies or fragments thereof, each possibly in encapsulated form or encapsulated as a mixture of the two. It may also comprise one (or more) micro-organism(s) that produce the two different antibodies or antibody fragments, either as monovalent or as polyvalent molecules. It may also comprise a combination of at least two micro-organism(s) that each produces one of the two different antibodies or antibody fragments. Finally, it may also comprise one or more micro-organism(s) that produce one of the two different antibodies or antibody fragments, whereby the other antibody or antibody fragment is added separately, optionally in encapsulated form.

It may be useful to employ a delivery system for the antibodies or fragments thereof to deliver them to the Gastro-intestinal tract, this can be effected by the use of encapsulates, such as those known in the food and pharmaceutical industries. Natural biopolymers may be used. Examples include Ca-alginate, carrageenan, gellan gum or gelatine. The delivery system may be an encapsulation method known in the art which will deliver the immunoglobulin or fragments thereof specifically to the gut. The encapsulate must therefore be able to survive until entry to the gut and then be released. Such a delivery system comprises a general protective system that protects the antibodies from degradation. Such techniques may include liposome entrapment, spinning disk and coacervation. Any trigger can be used to prompt the release of the encapsulated ingredient, such as pH change (enteric coating), mechanical stress, temperature, enzymatic activity. These techniques are expanded on in the article by Sebastien Gouin “Microencapsulation: industrial appraisal of existing technologies and trends” Food Science and Technology (2004) 15: 330-347. Preferably, an enteric coating is used. Additionally, the encapsulation method may allow the slow release of the antibody in the gut and/or stomach. This will enable a constant release of the antibody or functional fragment or equivalent over a set period of time.

Alternatively, according to another aspect of this embodiment the delivery system may comprise a (one or two) micro-organism, preferably transformed to be able to produce the antibodies or antibody fragments. This micro-organism(s) is independent from the antibodies or antibody fragments. The invention may comprise two or more different micro-organisms. The first is a probiotic micro-organism which does not form part of any delivery system for the antibodies or fragments thereof. The second is the micro-organism(s) which may form part of the delivery system. The former is referred to herein as the “probiotic micro-organism” and the latter as the “micro-organism”.

According to a particular aspect of the present invention there is provided a pharmaceutical preparation comprising a delivery system for delivering antibodies to the GIT wherein the antibodies are active in the gut and the delivery system comprises (a) micro-organism(s) transformed with antibodies or fragments thereof wherein the antibodies are heavy chain immunoglobulins of the “heavy chain antibodies” type or VHH fragments thereof, preferably derived from Camelids, most preferably llama heavy chain antibodies or fragments thereof, or domain antibodies (dAbs) of the heavy or light chains of immunoglobulins or fragments thereof and optionally independently a probiotic micro-organism.

Like the probiotic micro-organism, the micro-organism should preferably be able to survive passage in the GIT and should be active in the stomach/gut. Preferably, the micro-organism should be able to undergo transient colonization of the GIT; be able to express the gene in the GIT; and be able to stimulate the gut immune system.

Preferably, the micro-organism may also be a probiotic micro-organism with the above characteristics. In this case there will be two (or more) probiotic micro-organisms used according to the invention; one which is independent of the antibodies or fragments thereof and one which forms part of a delivery system therefor. Probiotics are defined as viable microbial food supplements which beneficially influence the host by improving its intestinal microbial balance in accordance to Fuller (1989) probiotics in man and animals, Journal of Applied Bacteriology 66, 365-378. If the probiotic micro-organism is a bacterium, it is preferred that it is a lactic acid bacterium.

Examples of other suitable probiotic micro-organisms include yeast such as Saccharomyces, Debaromyces, Kluyveromyces and Pichia, moulds such as Aspergillus, Rhizopus, Mucor and Penicillium and bacteria such as the genera Bifidobacterium, Propionibacterium, Streptococcus, Enterococcus, Lactococcus, Bacillus, Pediococcus, Micrococcus, Leuconostoc, Weissella, Oenococcus and Lactobacillus. Kluyveromyces lactis may also be used.

Specific examples of suitable probiotic micro-organisms are described in WO2006/056306 and include: Kluyveromyces lactis, Kluyveromyces fragilis, Pichia pastoris, Saccharomyces cerevisiae, Saccharomyces boulardii, Aspergillus niger, Aspergillus oryzae, Mucor miehei, Bacillus subtilis, Bacillus natto, Bifidobacterium adolescentis, B. animalis, B. breve, B. bifidum, B. infantis, B. lactis, B. longum, Enterococcus faecium, Enterococcus faecalis, Escherichia coli, Lactobacillus acidophilus, L. brevis, L. casei, L. delbrueckii, L. fermentum, L. gasseri, L. helveticus, L. johnsonii, L. lactis, L. paracasei, L. plantarum, L. reuteri, L. rhamnosus, L. sakei, L. salivarius, L. sanfranciscus, Lactococcus lactis, Lactococcus cremoris, Leuconostoc mesenteroides, Leuconostoc lactis, Pediococcus acidilactici, P. cerevisiae, P. pentosaceus, Propionibacterium freudenreichii, Propionibacterium shermanii and Streptococcus salivarius.

Particular probiotic strains are: Saccharomyces boulardii, Lactobacillus casei shirota, Lactobacillus casei immunitas, Lactobacillus casei DN-114 001, Lactobacillus rhamnosus GG (ATCC53103), Lactobacillus reuteri ATCC55730/SD2112, Lactobacillus rhamnosus HN001, Lactobacillus plantarum 299v (DSM9843), 25 Lactobacillus johnsonii La1 (1-1225 CNCM), Lactobacillus plantarum WCFS1, Bifidobacterium lactis HN019, Bifidobacterium animalis DN-173010, Bifidobacterium animalis Bb12, Lactobacillus casei 431, lactobacillus acidophilus NCFM, Lactobacillus reuteri ING1, Lactobacillus salivarius UCC118, Propionibacterium freudenreichii JS, Escherichia coli Nissle 1917.

Conveniently, the micro-organism may be a lactic acid bacterium. More, preferably, the micro-organism is chosen from either lactobacillus or bifidobacteria. Even more preferably, the micro-organism is Lactobacillus. Particularly, the Lactobacillus is Lactobacillus casei 393 pLZ15. Lactobacillus casei has recently been reidentified as Lactobacillus paracasei (Perez-Martinez, 2003). Another preferred Lactobacillus is Lactobacillus reutarii.

Alternatively, the micro-organism may be yeast. Suitable yeasts include the baker's yeast S. cerevisiae. Other yeasts like Candida boidinii, Hansenula polymorpha, Pichia methanolica and Pichia pastoris which are well known systems for the production of heterologous proteins and may be used in the present invention.

Filamentous fungi, in particular species from the genera Trichoderma and Aspergillus have the capacity to secrete large amounts of proteins, metabolites and organic acids into their culture medium. This property has been widely exploited by the food and beverage industries where compounds secreted by these filamentous fungal species have been used for decades.

A delivery system based on (probiotic) bacteria represents a safe and attractive approach and represents one of the cheapest antibodies production systems. The wide scale application of the micro-organism, preferably Lactobacillus, expressing antibodies is relatively easy and requires minimal handling and storage costs and economical.

Preferably, the micro-organism is transformed with an expression vector comprising the gene for the antibody. The expression vector may contain a constitutive promoter in order to express the antibodies or fragments thereof. Such a constitutive promoter will support in situ expression of antibodies by transformed lactobacilli persisting (at least for a short period) in the intestinal tract after administration. Alternatively, the promoter may be chosen to be active only in the GIT and/or stomach/gut i.e. suitable for GIT specific expression only. This will ensure expression and/or secretion of the llama heavy chain antibody or fragments thereof in the GIT, preferably the gut. Many constitutive promoters for lactobacilli are known in the art and an example of a promoter that is specifically inducible in the GIT is Pldh (Pouwels et al “Lactobacilli as vehicles for targeting antigens to mucosal tissues by surface exposition of foreign antigens” Methods in Enzymology (2001) 336:369-389).

The expression vectors described in the examples are able to replicate in the transformed lactobacilli and express the antibodies of fragments thereof. It will be understood that the present invention is not limited to these replication expression vectors only. The whole expression cassette can be inserted in a so-called “integration” plasmid, whereby the expression cassette will be integrated into the chromosome of the lactobacilli after transformation, as known in the art (Pouwels, P. H. and Chaillou, S. Gene expression in lactobacilli (2003) Genetics of lactic acid bacteria page 143-188). Thus, replicating or integrating vectors may be used in accordance with the invention.

When the delivery system comprises one or more micro-organism transformed with antibodies or fragments thereof the antibodies are expressed (displayed) and/or secreted in the gut. Hence, use of a micro-organism as the delivery system has the advantages that in vivo production of antibody fragments locally in the GIT circumvents the practical problem of degradation of orally administered antibodies in the stomach. Such a system based on probiotic bacteria represents a safe and attractive approach to delivering antibodies to the GIT. Hence, the wide scale application of the lactobacilli expressing antibodies is relatively easy and requires minimal handling and storage costs and economical. Furthermore, the probiotic bacteria will remain in the gut for longer and enable the constant production of the antibody to enable more constant protection against the enteropathogenic micro-organism. Preferably such a food product or pharmaceutical preparation comprises a micro-organism transformed to be able to produce the antibodies or fragments thereof, expresses a heterodimer of VHH3-VHH1 (SEQ ID No. 3-SEQ ID No. 1).

Advantageously the amount of the micro-organism in the delivery system in food products of the invention is between 10⁶ and 10¹¹ per serving or (for example if serving size is not known) between 10⁶ and 10¹¹ per 100 g of product, more preferred these levels are from 10⁸ to 10⁹ per serving or per 100 g of product.

The antibodies for use according to the present invention are preferably active in the gut/stomach, i.e. they must be functional and retain their normal activity including inactivating their target. The active antibodies according to the invention should bind to their target as normal, thus, the binding affinity of the antibody for the antigen should be as normal. Binding affinity is present when the dissociation constant is more than 105. Hence, the food product or pharmaceutical preparation according to the invention will be able to selectively address a specific disease or symptom of a disease. The disease or symptom to be treated or reduced will determine the choice of antibody.

It will be understood that when the product is a food product any antibody may be used. However, when the product is a pharmaceutical preparation heavy chain immunoglobulins or fragments thereof of the VHH or VNAR type or domain antibodies (dAbs) of the heavy or light chains of immunoglobulins or fragments thereof are preferred.

The antibodies or fragments thereof should have one or more of the following characteristics:
i) They show good binding affinity and the desired inhibition functionality (preferably) under the conditions present in the Gil tract; and
ii) They have good proteolytic stability in that they are stable against degradation by proteolytic enzymes.
iii) The antibodies should be thermostable which enables their inclusion in a variety of food products. The food products may be prepared in a process requiring pasteurization and it is preferred that the activity of the antibodies is largely maintained despite heat treatment.

The use of fragments or portions of a whole antibody which can nevertheless exhibit antigen binding affinity is also contemplated. Fragments should be functional fragments. A functional fragment of an immunoglobulin means a fragment of an immunoglobulin which fragment show binding affinity for an antigen and has the same biological activity as the full length sequence. Such fragments include Fab and (sc) Fv fragments. Binding affinity is present when the dissociation constant is more than 10exp5. Such a fragment can be advantageously used in therapy or prevention, for example, as it is likely to be less immunogenic and more able to penetrate tissues due to its smaller size.

Functional equivalents are also contemplated. A functional equivalent means a sequence which shows binding affinity for an antigen similar to the full length sequence. For example, additions, substitutions or deletions of amino acids which do not result in a change of functionality are encompassed by the term functional equivalents.

The antibody or fragment thereof should preferably be able to be expressed and secreted in the gut. Several assays are well known in the art which mimic GIT conditions and are used for instance to select suitable probiotics that can survive GIT conditions. A suitable assay for determining whether an antibody can survive the GIT conditions is described by Picot, A. and Lacroix, C. (International Dairy Journal 14 (2004) 505-515).

In order to determine whether an antibody will be suitable for use in the present invention the following test may be applied. The antibody produced is selected under specific conditions of low pH, preferably from 1.5 to 3.5, and in the presence of pepsin (a protease abundant in the stomach) to result in highly beneficial molecules that work well in the G/I tract and are suitable for use according to the present invention.

The antibody or fragment thereof may be naturally occurring or may be obtained by genetic engineering using techniques well known in the art. The antibody is selected to be active against viruses. The present application may be applicable to the management of enteropathogenic viruses. Management is understood to mean therapy and/or prophylaxis.

Enteropathogenic viruses may include, for example, Norovirus (Norwalk like virus), enteric adenovirus, Coronavirus, astroviruses, caliciviruses, and parvovirus. Rotavirus and the Norwalk family of viruses are the leading causes of viral gastroenteritis, however, a number of other viruses have been implicated in outbreaks. Most preferably, the present invention is directed to the management of rotaviral infection. The present application may also be used in the management of other non-enteropathogenic viruses like Hepatitis or SARS.

Preferably, heavy chain immunoglobulins or fragments thereof of the VHH or VNAR type or domain antibodies of the heavy or light chains of immunoglobulins or fragments thereof may be used in the present invention. Such heavy chain immunoglobulins of the VHH or VNAR type are obtained using techniques well known in the art. More preferably, the immunoglobulin or fragment thereof is derived from Camelids, most preferably llamas.

Van der Linden, R. H., et al. “Comparison of physical properties of llama VHH antibody fragments and mouse monoclonal antibodies” Biochim, Biophys. Acta (1990) 1431, 37-46 obtained heavy chain antibodies with a high specificity and affinity against a variety of antigens. Furthermore, heavy chain immunoglobulins are readily cloned and expressed in bacteria and yeast as shown in Frenken, L. G. J., et al. “Isolation of antigen specific Llama V_{HH} antibody fragments and their high level secretion by Saccharomyces cerevisiae”. J. Biotechnol. (2000) 78, 11-21. Methods for the preparation of such immunoglobulins or fragments thereof on a large scale comprising transforming a mould or a yeast with an expressible DNA sequence encoding the antibody or fragment are also described in patent application WO 94/25591 (Unilever). Finally, EP-A-0584421 describes heavy chain immunoglobulin regions obtained from Camelids.

The immunoglobulin or fragment thereof may be monovalent, multivalent (multispecific), i.e. bivalent, trivalent, tetravalent, in that it comprises more than one antigen binding site. The antigen binding sites may be derived from the same parent antibody or fragment thereof or from different antibodies which bind the same epitope. If all binding sites have the same specificity then a monospecific immunoglobulin-(fragment) is produced. Alternatively a multispecific immunoglobulin-(fragment) may be produced binding to different epitopes of the same antigen or even different antigens. It is preferred that the, or at least one of the, binding sites is directed to pathogens (or products thereof such as enzymes produced therefrom) found in the gastro-intestinal tract. If is further preferred that the immunoglobulin or fragment thereof binds to rotavirus and more preferably that it neutralises it.

The immunoglobulin or fragment thereof of the VHH- or VNAR-type, or domain antibodies (dAbs) of the heavy or light chains of immunoglobulins or fragments thereof, may be naturally occurring i.e. elicited in vivo upon immunizing an animal with the desired antigen or synthetically made, i.e. obtained by genetic engineering techniques.

Techniques for synthesising genes, incorporating them into micro-organism hosts and expressing genes in micro-organisms are well known in the art and the skilled person would readily be able to put the invention into effect using common general knowledge. The use of replicating or integrating vectors is contemplated.

According to one embodiment of the present invention, the food product or pharmaceutical preparation comprises antibodies which are heavy chain immunoglobulins or fragments thereof of the VHH- or VNAR-type, or domain antibodies (dAbs) of the heavy or light chains of immunoglobulins or fragments thereof which are active in the gut. According to one aspect of this invention the food product or pharmaceutical preparation comprises a delivery system for delivering the aforementioned antibodies to the GIT wherein the delivery system is a micro-organism and the immunoglobulins are llama derived antibodies or fragments thereof. In WO 06/056306 we have described that these transformed micro-organisms will express llama heavy chain antibodies or fragments thereof on their surface and are able to reduce the viral load, normalize the pathology and mitigate the diarrhea in an animal model of rotavirus infection. Furthermore, the llama heavy chain antibodies or fragments thereof were found to be very effective in reducing infection both in in vitro and in vivo models of rotavirus infection. Llama VHH antibody fragments, even in their monovalent form, have surprisingly been found to reduce the viral load, normalize the pathology and mitigate diarrhea during rotavirus infection.

In accordance with the present invention, there is provided a synergistic combination of at least two different antibodies or antibody fragments which are directed against a virus. It was surprisingly found that the combination of two different VHH fragments (e.g. VHH1 and VHH3) can give a synergistic activity against rotavirus. Examples of other synergistic combinations are: VHH1 and VHH 15, VHH 2 and VHH 3, VHH 4 and VHH 3, VHH 15 and VHH 2 and VHH 4 and VHH15. More generally, synergistic combinations consist of an antibody or antibody fragment competing with VHH 1 and an antibody or antibody fragment competing with VHH 3. On the basis of the present patent application, the skilled person will have no difficulty to find further synergistic combinations of antibodies or antibody fragments.

Particularly preferred llama derived VHH sequences having affinity of rotavirus are provided by this specification in the sequence listing, SEQ ID No's 1 to 21. Alternatively, VHH sequences having at least 70%, 80%, 85%, 90%, 95%, 98% or 99% amino acid identity with SEQ ID No. 1 and having affinity for a rotavirus particle or antigen are also preferred embodiments according to this invention. Alternatively, VHH sequences having at least 70%, 80%, 85%, 90%, 95%, 98% or 99% amino acid identity with any of the other SEQ ID Nos. 2-21 and having affinity for a rotavirus particle or antigen are also preferred embodiments according to this invention. VHH sequences may be derived from camellids, via immunization and/or by screening for affinity, but may also be derived from other mammalian species such as mice or humans and/or be camelized by amino acid substitutions, as described in the art. In another embodiment, the VHH sequences may be fused to yield multimeric units of 2, 3, 4, 5 or more VHH units, optionally linked via a spacer molecule. In another embodiment, several VHH sequences may be combined, either separately or in one multimeric molecule. Preferably the VHH sequences have different specificities, for instance VHH sequences may be combined to provide a wide spectrum of affinities for a particular pathogen. In a highly preferred embodiment, 2, 3, 4, 5 or more VHH sequences having affinity for any one of rotavirus strains Wa, CK5, Wal, RRV, CK5, G1P[8], G2P[4], G3P[8], G4P[8], G9P[6], or G9P[8].may be combined, as separate monomeric units or as combined units on a carrier, for instance on a probiotic bacterium and/or on a multimeric molecule.

Preferably the present invention provides a food product or pharmaceutical preparation according to the first aspect of the invention, comprising antibodies or antibody fragments having at least 80% amino acid sequence homology with VHH1 and VHH3 protein (SEQ ID No. 1 and SEQ ID No. 3 protein, respectively).

Preferably the present invention provides a food product or pharmaceutical preparation according to the first aspect of the invention, wherein the antibodies or antibody fragments recognise the same epitopes as VHH1 and VHH3. (SEQ ID No. 1 and SEQ ID No. 3, respectively).

Preferably a food product or pharmaceutical preparation according to the first aspect of the invention comprises a heterodimer of VHH3-VHH1 (SEQ ID No. 3-SEQ ID No. 1).

In WO 06/056306, we furthermore describe that llama heavy chain antibodies are suitable for administration in the GIT. Llama heavy chain antibodies were found to be highly resistant to protease degradation in the stomach and to withstand the acidic environment of the stomach. This is despite the fact that the proteolytic system in the GIT is more aggressive an environment than, for example encountered in the mouth.

Activity in the gut is hampered by proteolytic activity, including protease and peptidase. We have found that even more surprisingly the in vivo production or release of antibody fragments locally in the GIT circumvents the practical problem of degradation of orally administered antibodies in the stomach and gut.

When probiotic micro-organisms are chosen as the delivery system, we have found that these transformed micro-organisms will express llama heavy chain antibodies or fragments thereof on their surface and are able to reduce the viral load, normalize the pathology and mitigate the diarrhea in an animal model of rotavirus infection.

The llama heavy chain antibodies are then expressed by the micro-organism in the GIT. Expression of the llama derived VHH antibody fragment may be both on the surface of the micro-organism and/or as a secreted protein of the micro-organism. Preferably secreted forms of the VHH antibody fragment is in multimeric form to enhance aggregation and clearance of the viral load.

The food product or pharmaceutical preparation according to the invention comprising a synergistic combination of at least two different antibodies or antibody fragments may further comprise a probiotic micro-organism. This probiotic micro-organism may be used in either a viable or non-viable condition as desired. If the micro-organisms are to be used in a non-viable state then they may be rendered non-viable by any suitable means.

The probiotic micro-organism may be any suitable, edible, probiotic bacteria, mould or yeast and in particular may be of any of the types, including the preferred types, listed hereinabove for the micro-organism which forms a part of any delivery system for the antibodies or fragments thereof. Particularly preferred probiotic bacteria for use as the ‘independent probiotic micro-organism’ are Lactobacillus sp., especially Lactobacillus reutarii.

Advantageously the amount of the micro-organism in the delivery system in food products of the invention is between 10⁶ and 10¹¹ per serving or (for example if serving size is not known) between 10⁶ and 10¹¹ per 100 g of product, more preferred these levels are from 10⁸ to 10⁹ per serving or per 100 g of product. In some circumstances, it is advantageous of the total amount of micro-organism in the food product (i.e. the total of the amount of the micro-organism in the delivery system and the amount of the probiotic micro-organism which is independent from the antibodies or fragments thereof) is between 10⁶ and 10¹¹ per serving or (for example if serving size is not known) between 10⁶ and 10¹¹ per 100 g of product, more preferred these levels are from 10⁸ to 10⁹ per serving or per 100 g of product. The probiotic micro-organism may be added by any suitable means to the food product or pharmaceutical preparation.

### Food Products

Several food products may be prepared according to the invention, for example meal replacers, soups, noodles, ice-cream, sauces, dressing, spreads, snacks, cereals, beverages, bread, biscuits, other bakery products, sweets, bars, chocolate, chewing gum, diary products, dietetic products e.g. slimming products, (powder) drinks, complementary foods, syrups, etc. For some applications food products of the invention may also be dietary supplements, although the application in food products of the above type is preferred.

In all applications the transformed micro-organisms can be added as viable cultured (wet) biomass or as a dried preparation, still containing viable micro-organisms as known in the art.

The Table below indicates a number of products, which may be prepared according to the invention, and a typical serving size.

| Product | Serving Size | |
|---|---|---|
| margarine | 15 | g |
| ice-cream | 150 | g |
| dressing | 30 | g |
| sweet | 10 | g |
| bar | 75 | g |
| meal replacer drink | 330 | ml |
| Beverages (including | 200 | ml |
| powders to be used to | | |
| prepare a beverage) | | |

An alternative means of administration of the antibodies or fragments thereof (including a delivery system comprising a micro-organism transformed with antibodies or functional fragments thereof) comprises a dispensing implement for use with a food product which implement is coated on at least one surface with antibodies or anti-body fragments which are active in the gut. Optionally the antibodies or antibody fragments comprise a delivery system for delivering the antibodies or antibody fragments to the GIT.

For the above means of administration it is preferred that the delivery system comprises encapsulated antibodies or antibody fragments and/or that wherein the delivery system comprises a micro-organism transformed to be able to produce antibodies or fragments thereof.

The term dispensing implement covers tube, straws, knives, forks, spoons or sticks or other implements which are used to deliver a liquid or semi-solid food product to a consumer. The dispensing implement may also be used to deliver a solid food product to a consumer. This dispensing tube or straw is especially suitable for use with certain beverages where high or low pH and/or temperature means that direct addition of the micro-organism or antibody or antibody fragment to the beverage is not recommended.

The dispensing implement can also be used when the delivery system of the invention comprises encapsulated antibodies or fragments thereof or even with antibodies or fragments thereof per se.

After the dispensing implement is coated with the relevant components according to the above, the implement is stored in an outer envelope which is impermeable to moisture and other contamination. The coating material which contains these particles is non-toxic to humans and to bacteria and can be an oil such as corn oil or a wax. This aspect is described in U.S. Pat. No. 6,283,294. Once the dispensing implement containing these components penetrates the beverage or semi-solid food product, the particles are integrated into the food product, giving a desirable dose of the antibodies or fragments thereof and the (probiotic) micro-organisms with a serving of the product.

Preferably, the components above to be coated onto the implement may be suspended in water which is then applied to the dispensing implement and evaporated. By using this method the dispensing implement will have a coating of the components which can then be released when the dispensing implement comes into contact with the liquid or semi-solid food product.

A still further embodiment of the invention relates to a method for making a food product or pharmaceutical preparation according to the invention.

If it is desired that the micro-organism and/or the optional probiotic micro-organism is/are alive in the product, for example, if the product is heated during processing, the micro-organism has to be added after the heating step (post-dosing). However, if a product is fermented with the micro-organism, a heating step after the fermentation may not be acceptable. If the product is a liquid product, administration of the micro-organism could take place by use of a dispensing implement such as a drinking straw.

A further embodiment of the invention relates to the use of the food product or pharmaceutical preparation according to the invention to deliver health benefits to the gut of a subject after administration. Such health benefits include the specific health benefit the antibody may provide. The micro-organism itself used in any delivery system may also provide several health effects for example relating to gut well being such as IBS (Irritable Bowel Syndrome), reduction of lactose maldigestion, clinical symptoms of diarrhea, immune modulation, anti-tumor activity, adjuvant effects and enhancement of mineral uptake.

The food product or pharmaceutical preparation according to the present invention may be suitable for the management, including treatment or prophylaxis of infections caused by enteropathogenic bacteria or viruses. Other antibodies which may be incorporated into the invention will be able to provide a multitude of other health benefits.

Hence the present invention provides a food product or pharmaceutical preparation according to the first aspect of the invention or made according to the second aspect of the invention for use as a medicament. Preferably the food product or pharmaceutical preparation according to the first aspect of the invention or made according to the second aspect of the invention is for use to combat enteropathogenic micro-organisms, in particular viral infections. This involves use of the food product or pharmaceutical preparation according to the first aspect of the invention or made according to the second aspect of the invention to combat enteropathogenic micro-organisms, in particular viral infections.

Preferably the food product or pharmaceutical preparation according to the first aspect of the invention or made according to the second aspect of the invention, comprises antibodies or antibody fragments that are llama heavy chain antibodies or antibody fragments to deliver an anti-diarrheal effect. This involves use of the food product or pharmaceutical preparation according to the first aspect of the invention or made according to the second aspect of the invention, wherein the antibodies or antibody fragments are llama heavy chain antibodies or antibody fragments to deliver an anti-diarrheal effect.

Preferably the food product or pharmaceutical preparation according to the first aspect of the invention or made according to the second aspect of the invention is for use in the management of rotavirus infection. This involves use of the food product or pharmaceutical preparation according to the first aspect of the invention or made according to the second aspect of the invention in the management of rotavirus infection.

The present invention also provides a method of delivering health benefits to the gut of a subject comprising administering the food product or pharmaceutical preparation according to the first aspect of the invention or made according to the second aspect of the invention to a subject in need thereof.

The present invention is based on the finding that heavy chain immunoglobulins or fragments thereof of the VHH- or VNAR-type, or domain antibodies (dAbs) of the heavy or light chains of immunoglobulins or fragments thereof, of the invention may be used in the therapy or prophylaxis of infection by enteropathogenic micro-organisms. Furthermore, the immunoglobulins or fragments thereof of the VHH- or VNAR-type, or domain antibodies (dAbs) of the heavy or light chains of immunoglobulins or fragments thereof, may be used in the therapy or prophylaxis of viral gastroenteritis or diarrhea caused by the enteropathogenic microorganism rotavirus.

A further advantage of the present invention is that the use of food products or pharmaceutical preparations comprising probiotic micro-organisms expressing immunoglobulins or fragments thereof of the VHH- or VNAR-type, or domain antibodies (dAbs) of the heavy or light chains of immunoglobulins or fragments thereof, enables the micro-organism used as part of any delivery system, for example Lactobacillus, to provide the normal health benefits associated therewith, together with the prophylactic/therapeutic benefits in the management of the infection to be treated. This “dual effect” therapy provides greater health benefits to the subject than that known in the art.

In accordance with another embodiment of the present invention, the heavy chain immunoglobulins or fragments thereof of the VHH type are derived from camelids, including llama and camels. Many llama derived heavy chain antibody fragments have been disclosed in the art. More preferred is the heavy chain immunoglobulin or fragment thereof which shows binding affinity with a dissociation constant of at least 10 exp 5 for rotavirus, especially rotavirus strains Wa, CK5, Wal, RRV, CK5, G1P[8], G2P[4], G3P[8], G4P[8], G9P[6], or G9P[8].

It is known that llama heavy chain antibodies may be effective in the management of rotavirus infection. When the antibodies used are llama heavy chain antibodies, the health benefit delivered will include an anti-diarhoeal effect. Hence, llama heavy chain antibodies can be used in the management of rotavirus infection, including the therapy or prophylaxis of rotavirus infection. We have found that llama VHH antibody fragments can reduce the viral load, normalize the pathology and mitigate diarrhea during rotavirus infection. Rotavirus continues to be the single most common cause of infantile diarrhea in the world and most children get infected during the first 5 years of life. In developing countries, rotavirus induced diarrhea may cause 600,000 to 870,000 deaths each year and in developed countries, rotavirus disease accounts for immense economic loss.

It will be understood that the food product or pharmaceutical preparation can be administered in order to deliver a health benefit to the subject and/or to combat a specific disease or infection. The choice of the antibodies will depend on the disease to be treated.

Preferably, the micro-organism is transformed with an expression vector comprising the gene for the llama heavy chain antibody or fragment thereof. Either an integrating or a replicating vector may be used.

If encapsulation is chosen as the delivery system, the encapsulation method should survive passage to the stomach through the GIT and should be able to provide a sustained release of the antibody over a set period of time. This will ensure that the llama heavy chain antibody or fragment is delivered over time to the stomach. Llama heavy chain antibodies or heavy chains thereof are particularly suitable for this encapsulation method due to their ability to survive in the gut when released.

Specifically, the antibodies which form part of any delivery system may be delivered to the GIT using a micro-organism transformed with llama heavy chain antibodies comprising the steps of i) transforming the micro-organism with the gene encoding llama heavy chain antibodies; and ii) administering the transformed micro-organism to the GIT of the human or animal in need of therapy.

The invention will now be further illustrated by the description of suitable embodiments of the preferred food products for use in the invention. It is believed to be well within the ability of the skilled person to use the teaching provided therewith to prepare other products of the invention.

### Margarines and Other Spreads

Typically these are oil in water or water in oil emulsions, also spreads which are substantially fat free are covered. Typically these products are spreadable and not pourable at the temperature of use e.g. 2-10 C. Fat levels may vary in a wide range e.g. full fat margarines with 60-90 wt % of fat, medium fat margarines with 30-60 wt % of fat, low fat products with 10-30 wt % of fat and very low or fat free margarines with 0 to 10 wt/0 of fat.

The fat in the margarine or other spread may be any edible fat, often use are soybean oil, rapeseed oil, sunflower oil and palm oil. Fats may be used as such or in modified form e.g. hydrogenated, esterified, refined etc. Other suitable oils are well known in the art and may be selected as desired. The pH of a margarine or spread may advantageously be from 4.5 to 6.5. Examples of spreads other than margarines are cheese spreads, sweet spreads, yogurt spreads etc.

Optional further ingredients of spreads may be emulsifiers, colourants, vitamins, preservatives, emulsifiers, gums, thickeners etc. The balance of the product will normally be water.

A typical size for an average serving of margarine or other spreads is 15 grams. Preferred VHH-producing Lactobacillus (or other VHH producing micro-organism) in the margarine or spread are 10⁶ and 10¹¹ per serving most preferred 10⁸ to 10¹⁰ per serving. The Lactobacillus strain has to be added aseptically after the heating steps in the process. Alternatively, encapsulated VHH's may be added to these food products. Preferably between 25 and 5000 μg per serving is added, more preferably between 50 and 500 μg are added per serving. Most preferably two or three servings are given each day.

### Frozen Confectionary Products

For the purpose of the invention the term frozen confectionery product includes milk containing frozen confections such as ice-cream, frozen yoghurt, sherbet, sorbet, ice milk and frozen custard, water-ices, granitas and frozen fruit purees.

Preferably the level of solids in the frozen confection (e.g. sugar, fat, flavouring etc) is more than 3 wt %, more preferred from 10 to 70 wt %, for example 40 to 70 wt %.

Ice-cream will typically comprise 2 to 20 wt % of fat, 0 to 20 wt % of sweeteners, 2 to 20 wt % of non-fat milk components and optional components such as emulsifiers, stabilisers, preservatives, flavouring ingredients, vitamins, minerals, etc, the balance being water. Typically ice-cream will be aerated e.g. to an overrun of 20 to 400%, more general 40 to 200% and frozen to a temperature of from −2 to −200 C, more general −10 to −30 C. Ice-cream normally comprises calcium at a level of about 0.1 wt %.

A typical size of an average serving of frozen confectionary material is 150 grams. Preferred Lactobacillus (or other VHH producing micro-organism) levels are from 10⁶ and 10¹¹ per serving, more preferred these levels are from 10⁷ to 10¹⁰ per serving most preferred 10⁸ to 10⁹ per serving. The Lactobacillus strain has to be added aseptically after the heating steps in the process. Alternatively, encapsulated VHH's may be added to these food products. Preferably between 25 and 5000 μg per serving is added, more preferably between 50 and 500 μg are added per serving. Most preferably two or three servings are given each day.

Beverages, for example Tea Based Products or Meal Replacers

Lactobacillus can advantageously be used to beverages for example fruit juice, soft drinks etc. A very advantageous beverage in accordance to the invention is a tea based product or a meal replacers drink. These products will be described in more detail herein below. It will be apparent that similar levels and compositions apply to other beverages comprising vitamin Lactobacillus bacteria.

For the purpose of this invention the term tea based products refers to products containing tea or tea replacing herbal compositions e.g. tea-bags, leaf tea, herbal tea bags, herbal infusions, powdered tea, powdered herbal tea, ice-tea, ice herbal tea, carbonated ice tea, carbonated herbal infusion etc.

Typically some tea based products of the invention may need a preparation step shortly before consuming, e.g. the making of tea brew from tea-bags, leaf tea, herbal tea bags or herbal infusions or the solubilisation of powdered tea or powdered herbal tea. For these products it is preferred to adjust the level of Lactobacillus in the product such that one serving of the final product to be consumed has the desired levels of Lactobacillus as described above.

For ice-tea, ice herbal tea, carbonated ice tea, carbonated herbal infusions the typical size of one serving will be 200 ml or 200 grams.

Meal replacer drinks are typically based on a liquid base which may for example be thickened by means of gums or fibres and whereto a cocktail of minerals and vitamins are added. The drink can be flavoured to the desired taste e.g. fruit or choco flavour. A typical serving size may be 330 ml or 330 grams.

Both for tea based beverages and for meal replacer drinks, preferred Lactobacillus levels are 10⁶ and 10¹¹ per serving, more preferred these levels are form 10⁷ to 10¹⁰ per serving most preferred 10⁹ to 10⁹ per serving. Alternatively, encapsulated VHH's may be added to these food products. Preferably between 25 and 5000 μg per serving is added, more preferably between 50 and 500 μg are added per serving. Most preferably two or three servings are given each day.

For products which are extracted to obtain the final product, generally the aim is to ensure that one serving of 200 ml or 200 grams comprises the desired amounts as indicated above. In this context, it should be appreciated that normally only part of the Lactobacillus present in the tea based product to be extracted will eventually be extracted into the final tea drink. To compensate for this effect generally it is desirable to incorporate into the products to be extracted about 2 times the amount as is desired to have in the extract. For leaf tea or tea-bags typically 1-5 grams of tea would be used to prepare a single serving of 200 mls.

If tea-bags are used, the Lactobacillus may advantageously be incorporated into the tea component. However it will be appreciated that for some application it may be advantageous to separate the Lactobacillus from the tea, for example by incorporating it into a separate compartment of the tea bag or applying it onto the tea-bag paper. Alternatively, the micro-organism may be administered in dried form through the use of a straw, spoon or stick with a coating of dried micro-organism.

### Salad Dressings or Mayonnaise

Generally dressings or mayonnaise are oil in water emulsions, the oil phase of the emulsion generally is 0 to 80 wt % of the product. For non fat reduced products the level of fat is typically from 60 to 80%, for salad dressings the level of fat is generally 10-60 wt %, more preferred 15-40 wt %, low or no fat dressings may for example contain triglyceride levels of 0, 5, 10, 15% by weight.

Dressings and mayonnaise are generally low pH products having a preferred pH of from 2-6.

Dressings or mayonnaise optionally may contain other ingredients such as emulsifiers (for example egg-yolk), stabilisers, acidifiers, biopolymers, bulking agents, flavours, colouring agents etc. The balance of the composition is water which could advantageously be present at a level of 0.1 to 99.9 wt %, more general 20-99 wt %, most preferred 50 to 98 wt %.

A typical size for an average serving of dressings or mayonnaise is 30 grams. Preferred levels of Lactobacillus in such products would be 10⁶ and 10¹¹ per serving, more preferred these levels are from 10⁷ to 10¹⁰ per serving most preferred 10⁸ to 10⁹ per serving. The Lactobacillus strain has to be added aseptically after the heating steps in the process. Alternatively, encapsulated VHH's may be added to these food products. Preferably between 25 and 5000 μg per serving is added, more preferably between 50 and 500 μg are added per serving. Most preferably two or three servings are given each day.

### Meal Replacer Snacks or Bars

These products often comprise a matrix of edible material wherein the Lactobacillus can be incorporated. For example the matrix may be a fat based (e.g. couverture or chocolate) or may be based on bakery products (bread, dough, cookies etc) or may be based on agglomerated particles (rice, grain, nuts, raisins, fruit particles).

A typical size for a snack or meal replacement bar could be 20 to 200 g, generally from 40 to 100 g. Preferred levels of Lactobacillus in such products would be 10⁶ and 10¹¹ per serving, more preferred these levels are from 10⁷ to 10¹⁰ per serving most preferred 10⁸ to 10¹⁹ per serving. The Lactobacillus strain has to be added aseptically after the heating steps in the process. Alternatively, encapsulated VHH's may be added to these food products. Preferably between 25 and 5000 μg per serving is added, more preferably between 50 and 500 μg are added per serving. Most preferably two or three servings are given each day.

Further ingredients may be added to the above product such as flavouring materials, vitamins, minerals etc.

For each of the above food products, the amount of Lactobacillus per serving has been given as a preferred example. It will be understood that alternatively any suitable micro-organism or virus may be present at this level.

### Lemonade Powder

Lactobacillus can also be used in dry powders in sachets, to be dissolved instantly in water to give a refreshing lemonade. Such a powder may have a food-based carrier, such as maltodextrin or any other. Optional further ingredients may be colourants, vitamins, minerals, preservatives, gums, thickeners etc.

Preferred VHH-producing Lactobacillus in the lemonade powder are 10⁶ and 10¹¹ per serving most preferred 10⁸ to 10¹⁰ per serving. The Lactobacillus strain has to be sprayed on the carrier in such a way that it is kept alive, according to methods known by those skilled in the art. Alternatively, encapsulated VHH's may be added to these food products. Preferably between 25 and 5000 μg per serving is added, more preferably between 50 and 500 μg are added per serving.

In all the above products the transformed micro-organism can be added as viable cultured (wet) biomass or as a dried preparation, still containing the viable micro-organisms as known in the art.

### BRIEF DESCRIPTION OF THE FIGURES

In the detailed description of the embodiments of the invention, reference is made to the following figures.

FIG. 1A shows in-vitro RRV inhibition assay. 9 microgram of VHH fragments were incubated with RRV which was subsequently used for infection of MA104 cells. The cell lysates were probed for the presence of rotavirus VP6 protein. (lane 1:VHH1, lane 2: VHH3, lane 3: VHH1+VHH3), lane 4: Irrelevant VHH, lane 5: uninfected cells, lane 6: infection only. VHH1 and VHH3 proteins in combination reduced the expression of VP6 in infected cells by 87.5% compared to 50% and 75% respectively for VHH1 and VHH3 as calculated by densitometric analysis.

FIG. 1B shows the results of second in-vitro RRV inhibition assay. The cell lysates were probed for the presence of rotavirus NSP4 protein. M=Molecular weight marker, lane 2=VHH1, 3=VHH2, 4=VHH3, 5=Irrelevant VHH, 6=Infection only, 7=VHH1+VHH3, 8=VHH2+VHH3, 9=VHH1+Irrelevant VHH, 10=no infection, 11=VHH1, 12=VHH2, 13=VHH15, M=Molecular weight marker, 15=Infection only, 16=VHH1+VHH15, 17=VHH2+VHH15, 18=no infection

FIG. 1C shows the results of a third in-vitro RRV inhibition assay. The cell lysates were probed for the presence of rotavirus NSP4 protein. M=Molecular weight marker, 2=VHH1—10 microliter, 3=VHH1—5 microliter, 4=VHH3—10 microliter, 5=VHH3—5 microliter, 6=VHH 1+VHH3—10 microliter, 7=VHH1+VHH3—5 microliter, 8=Irrelevant VHH 10 microliter, 9=Irrelevant 5 microliter, 10=Irr 2 microliter, 11=VHH2—10 microliter, 12=VHH2—5 microliter, 13=VHH15—10 microliter, 14=VHH15—5 microliter, M=Molecular weight marker, 16=VHH2+VHH15—10 microliter, 17=VHH2+VHH15—5 microliter, 18=Irrelevant VHH—5 microliter, 19=Irr—2 microliter

FIG. 2A shows the design of the expression cassettes in plAV7 for expression of anchored VHH monomers and dimers in lactobacilli. The expression cassettes were cloned in the plAV7 plasmid vector.

FIG. 2B shows the expression of VHH monomers and dimers by lactobacilli. Transformed lactobacilli were stained for the presence of the E-tag and analyzed by flow cytometry. Lactobacilli expressing surface anchored VHH1 (1, black), VHH3 (2, pink), VHH1-VHH1 (3, light blue), VHH3-VHH3 (4, orange), VHH3-VHH1 (5, dark blue), non-transformed lactobacilli (6, green).

FIG. 2C is the same plot as FIG. 2B, in three-dimensional form.

FIG. 2C shows the expression of VHH monomers and dimers by lactobacilli. Transformed lactobacilli were stained for the presence of the E-tag and analyzed by flow cytometry. Lactobacilli expressing surface anchored VHH1 (1), VHH3 (2), VHH1-VHH1 (3), VHH3-VHH3 (4), VHH3-VHH1 (5), non-transformed lactobacilli (wild type) (6).

FIG. 3 shows an estimation of avidity of different lactobacilli constructs to bind rotavirus by flow cytometry. Transformed lactobacilli were incubated with RRV and the samples analysed by flow cytometry. Lactobacilli expressing cell surface anchored VHH3-VHH1 protein is the best binder.

FIG. 4A shows the prophylactic administration of different lactobacilli expressing VHH fragments in reducing diarrhea. Lactobacilli expressing VHH3-VHH1 reduce diarrhea prevalence.

FIG. 4B shows the therapeutic administration of different lactobacilli expressing VHH fragments in reducing diarrhea. Lactobacilli expressing VHH3-VHH1 reduce diarrhea prevalence.

FIG. 5 shows the virus load in intestinal sections from different treatment groups.

Prophylactic administration of VHH1 and VHH3-VHH1 expressing lactobacilli reduces virus load. MIX is an equimolar mixture of VHH1 anchor and VHH3 anchor expressing lactobacilli.

The invention will now be further illustrated by means of the following examples.

### EXAMPLE 1

### Competition ELISA

In order to determine whether the anti-rotavirus fragments bind to the same or different epitopes on the virus particle a competition ELISA was performed. For this a 96 well plate was coated with virus particles. Purified VHH1 was biotinylated by adding NHS-biotin (N-Hydroxysuccinimidobiotin in DMSO, Sigma H-1759) in a molar ratio of 20:1 (NHS-biotin: llama fragment). After incubating on rotator for 2 hours at room temperature unbound biotin was removed by dialysis against PBS. The VHH1 fragment was mixed with other VHH fragments like VHH2, 3, 4, 15.

Inactivated Rotavirus G3 (CK5, 1×107 pfu/ml) was diluted 10 times in PBS and 100 μl/well was coated overnight at 4° C. onto a Nunc Maxisorp 96-well plate. The plate was blocked using 200 μl/well of 4% Marvel (skimmed milk powder) in PBS for 30 min with shaking. Then 50 μl of anti-Rota VHH sample, diluted in sample buffer (2% Marvel, 0.05% Tween-20 in PBS), was mixed with 50 μl of 250 ng/ml biotinylated anti-Rota VHH1 in sample buffer and transferred to the coated plate. After incubating for 1 hour at room temperature with shaking the plate was washed 3 times with wash buffer (0.05% Tween-20 in PBS). After washing, the plate was incubated with 100 μl/well horseradish conjugated steptavidin (diluted 1/1000 in sample buffer) for 1 hour at room temperature with shaking. The plate was washed 3 times with washing buffer and 3 times with deionised water. Colour development was started by adding 100 μl/well substrate (TMB/H2O2). The reaction was stopped with 50 μl/well of 1 M H2SO4 and OD450 was measured using a microtiterplate reader (Spectramax, Molecular devices).

### In-Vitro Rotavirus Inhibition Assay

In an initial experiment, VHH1 and VHH3 were produced in Saccharomyces cerevisae as described before [10]. MA104 cells were seeded in 24 well plates a day before infection at 1×105 cells /ml in DMEM with 5% FCS. 105 FFU of trypsinized rotavirus was added to dilutions of purified VHH1, VHH3 or a combination of both, prepared in OptiMEM to a final volume of 50 μl. A VHH fragment directed against an azo dye was used as an irrelevant control. The virus was incubated with the antibodies at RT for 15 minutes and used for infection of MA104 cells after adjusting the volume to 250 μl with DMEM for 1 hr at 37° C. After removing the virus, the cells were washed with DMEM, supplemented with DMEM+10% FCS and cultured for 14 hrs at 37° C. with 5% CO2. Cells were lysed and the extracts boiled with SDS loading dye for protein gels. 12% SDS-PAGE gels were cast and the proteins were separated by electrophoresis (Bio-Rad Laboratories). The proteins were electro-blotted on to nitrocellulose membrane using wet transfer (Bio-Rad Laboratories). The VP6 protein of rotavirus was detected using rabbit anti-VP6 antisera (1:1000) kindly provided by Dr Lennart Svensson (Karolinska Institute, Stockholm, Sweden)., followed by anti-rabbit HRP conjugated antibodies (DAKO A/S) (1:1000). The reaction was developed using the ECL chemiluminescence kit (GE Healthcare). The expression level of VP6 protein was estimated by desitomery.

In a second experiment, VHH1, VHH2 (cross reacting with VHH1), VHH3 and VHH15 (cross reacting with VHH3) were produced in Saccharomyces cerevisae as described before [10]. MA104 cells were grown in 24 wells plates at 10⁵cells/well. 20 μg antibody fragments were mixed with 10⁵ FFU of RRV and incubated at RT for 30 minutes. For combinations of different fragments 10 μg+10 μg were used. The volume was increased to 250 μl using serum free media and added to MA104 cells plated in a 24 well plate (please refer to the manuscript for further information on the technique). The virus was left on cells for 1 hr after which the supernatant was removed and the cells left to grow in the incubator overnight. After 14 hrs, the cells were lysed and western blot conducted for RRV NSP4 protein.

### Generation of Transformed Lactobacilli

The VHH1 and VHH3 fragments were selected from a llama immune library generated against the rhesus rotavirus strain RRV. The selection of the VHH1 fragment has been described in detail previously [10]. The VHH3 fragment was selected from the same library but was not included in the previous studies as the production rate of VHH3 is low in yeast. The genes encoding the VHH fragments were fused to an E-tag encoding gene and cloned in the plAV7 plasmid vector for expression in lactobacilli using the APF promoter and signal peptide (Marcotte et al. unpublished data). Dimers of VHH fragments (VHH1-VHH1, VHH3-VHH3 or VHH3-VHH1) were generated by fusing the two fragments, end to end, by PCR. For bacterial surface expression, an anchor sequence (the last 244 amino acids of the proteinase P protein of L. casei), was introduced after the E-tag encoding gene. Transformation of L. paracasei (previously named L. caseiATCC 393 pLZ15-) [11] was performed as described previously [8]. Lactobacilli transformed with the pLP502 plasmid vector encoding a llama antibody fragment against the SAI/II protein of Streptococcus mutans served as an irrelevant control [8]. The transformed lactobacilli were cultured in MRS broth containing 5 μg/ml erythromycin.

### Flow Cytometry

Lactobacilli were grown to an OD600 of 1 and were stained with a 1:200 dilution of a mouse anti E-tag monoclonal antibody (Amersham Biosciences) for 30 minutes on ice. Anti-mouse Cy2 conjugate (1:200) was used as secondary antibody (Jackson Immunoresearch Laboratories). The samples were analyzed using a FACS Calibur machine (Becton Dickinson). To ascertain binding to RRV, Lactobacilli grown to an OD600 of 0.8 were incubated with a 10 fold excess of RRV. The lactobacilli were then incubated with a 1:200 dilution of rabbit anti-rotavirus serum, followed by a 1:200 dilution of anti-rabbit PE conjugate antibody (Jackson Immunoresearch Laboratories). Incubations were performed on ice for 30 minutes. The lactobacilli were fixed using 2% paraformaldehyde and analyzed using a FACS Calibur machine (Becton Dickinson).

### Fluorescence Microscopy

MA104 cells were seeded on chamber slides (Becton Dickinson) a day before infection at 1×10⁵ cells /ml in DMEM with 5% FCS. On the day of infection, lactobacilli were grown to an OD600 of 0.8 and 50 μl of the culture was incubated with a 100 fold excess of trypsin activated RRV in a final volume of 100 μl for 20 minutes on ice. After adjusting the volume to 500 μl with OptiMEM, the mixture was used for infection of the cells for 1 hr at 37° C. and 5% CO2. The cells were washed and supplemented with DMEM with 10% FCS and incubated for 14 hrs at 37° C. and 5% CO2. The cells were fixed with chilled methanol for 10 minutes at RT and washed with PBS. Double immunofluorescent staining was performed to detect lactobacilli (using anti-E-tag antibodies) and rotavirus VP6 protein (using rabbit anti-VP6 antisera) which constitutes 50% of total protein in the rotavirus virions and also accumulates in the infected cells.

### In-Vivo Test

All animal experiments were approved by the local ethical committee of the Karolinska Institutet at Karolinska University Hospital, Huddinge. In-vivo tests were performed as described previously [8]. Briefly, purified VHH fragments or lactobacilli were administered to pups once daily in a 10 μl volume, starting on day −1 (for prophylactic treatment) and continuing until day 3. Infections were made orally on day 0 using 2*10⁷ ffu RRV (20 diarrhea doses (DD50)), a dose which causes diarrhea in more than 90% of inoculated animals. For therapeutic intervention, the first dose of VHH fragments or lactobacilli were administered to pups 2 hrs after infection and then continued once daily until day 3. Occurrence of diarrhea was recorded daily until day 4. Pups were euthanized on day 4 and sections of small intestine were stabilized in RNAlater® (QIAGEN) for RNA isolation.

### Statistics

Diarrhea in the pups was assessed on the basis of consistency of faeces. Watery diarrhea was given a score of 2 and loose stool was given a score of 1, no stool or normal stool was given a score of 0. Presence or absence of diarrhea was compared among the treatment groups in a day-wise manner by Fischer's exact test and was presented as percentage diarrhea in graphs. Severity was defined as the sum of diarrhea scores for each pup during the course of the experiment (severity=Σ diarrhea score (day 1+day 2+day 3+day 4)) and duration was defined as the total sum of days with diarrhea. Both severity and duration were analyzed by Kruskal-Wallis and Dunn tests. Differences in the intestinal virus load as assessed by real-time PCR were analyzed using the Mann-Whitney test.

### Quantitative RT-PCR for Estimation of Virus Load

Total cellular RNA was isolated from small intestinal tissue, treated with RNase-free DNase® (QIAGEN) and analyzed by real-time PCR using the EZ RT-PCR® core reagent kit (PE Applied C Biosystems). Rotavirus vp7 mRNA, or viral genomic RNA, was amplified at 58 (ABI 7000 cycler, Applied Biosystems) in the presence of 600 nM primers, 300 nM probe, and 5 mM Mn, to generate a 121-bp-long amplicon as described previously [8]. The RNA samples from each animal were normalized against expression of the GAPDH gene [12]. The presence of less than 10 copies of vp7 RNA was defined as clearance of infection.

### Results

### Crossreativity

Using a competition ELISA it was found that VHH1 binds to the same epitope as VHH 2 and 4 and VHH3 binds to the same epitope as VHH15.

### Combination of VHH1 and VHH3 Effectively Inhibits Rotavirus Infection In-Vitro

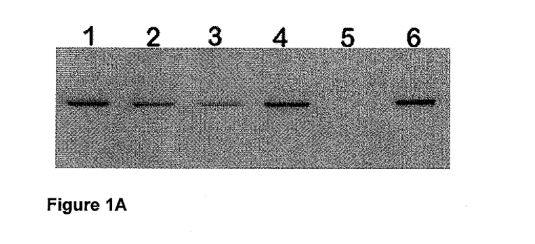

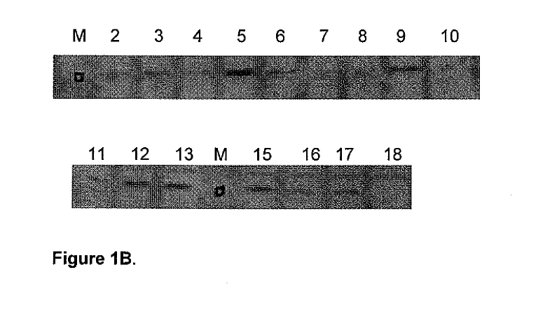

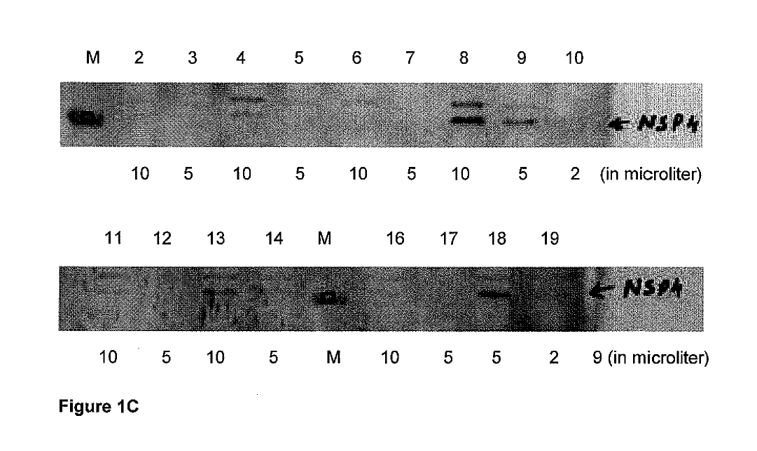

A dose of 9 μg (0.7 nmoles) of VHH1 or VHH3 proteins could reduce virus infection in MA104 cells by 50% and 75% respectively as judged by expression of VP6 protein. An irrelevant VHH fragment did not afford any protection against rotavirus infection. The combination of VHH1 and VHH3 (total dose of 9 μg representing 4.5 μg of each) was highly efficacious at inhibiting rotavirus infection and reduced VP6 expression by 87.5% (FIG. 1A). The protection achieved by the combination of VHH1 and VHH3 is 40% better than theoretical protection afforded additively by 4.5 μg of VHH1 and VHH3 (62.5%, 25% from VHH1 and 37.5% from VHH3). Apparently, the combination of VHH1 and VHH3 fragment acts synergistically against rotavirus infection.

The results of the second experiment (FIG. 1B) show that the combination of VHH1 and VHH3 and combination of VHH2 and VHH15 is superior at reducing infection compared to either fragment individually. In this case the viral NSP4 protein was detected using a rabbit anti NSP4 polyclonal antiserum. This serum was obtained by immunisation of rabbits with a synthetic peptide covering amino acid 114-134 of NSP4. This serum was also a gift of Dr Lennart Svensson (Karolinska Institute, Stockholm, Sweden).

Following a third experiment, comparable to the second, the reduction in rotavirus infectivity while using combination of VHH1 and VHH3 was quantified by densitometric analysis of Western blot developed for rotavirus NSP4 protein. Here cell extracts were loaded on wells in a 12% SDS gel in different amounts (10 μl, 5 μl or 2 μl) and subsequent to transfer, the blot was developed for rotavirus NSP4 protein.

From the image (FIG. 10) it can be seen that the irrelevant VHH protects poorly against infection. Approximately, 2 μl of cell extracts from irrelevant VHH group has the same amount of NSP4 as 5 μl extract from VHH1, VHH2, VHH3 or VHH15 group which again is similar to the NSP4 signal received from 10 μl of cell extracts from the VHH1 and VHH3 or VHH2 and VHH15 combination group.

From this it can be concluded that if the irrelevant VHH is said to have 100% infection, VHH1, 2, 3 and 15 have 40% infection and the combination groups have 20% infection. The ‘synergistic’ effect of the combinations, therefore is 50% better over a simple additive interaction. From a duplicate (forth) experiment a 40% increase in efficacy attributable to synergistic interaction was found.

TABLE 1

| | | | Highest % diarrhea | Duration ± | |
|---|---|---|---|---|---|
| 10 μg dose of | Regimen | n | prevalence | SE | Severity ± SE |
| VHH1 | prophylactic | 8 | 38 | 0.62 ± 0.26* | 0.87 ± 0.39* |
| VHH3 | prophylactic | 7 | 43 | 0.86 ± 0.34 | 1.00 ± 0.44 |
| VHH1 + VHH3 | prophylactic | 8 | 12 | 0.12 ± 0.12*** | 0.12 ± 0.12*** |
| Untreated | prophylactic | 8 | 100 | 2.00 ± 0.00 | 3.00 ± 0.26 |
| VHH1 | therapeutic | 7 | 43 | 0.71 ± 0.36 | 1.00 ± 0.48 |
| VHH3 | therapeutic | 5 | 60 | 1.00 ± 0.45 | 1.20 ± 0.58 |
| VHH1 + VHH3 | therapeutic | 6 | 33 | 0.50 ± 0.22 | 0.50 ± 0.22 |
| Untreated | therapeutic | 6 | 83 | 2.00 ± 0.25 | 2.33 ± 0.33 |

### Combination of VHH1 and VHH3 Purified Protein In Vivo

Prophylactic administration of 10 μg of VHH1 or VHH3 partially protected mice from diarrhea with a reduction of 62% and 57% in prevalence in the respective groups. A daily 10 μg dose of combination of VHH1 and VHH3 proteins (representing 5 μg each of either fragment) strongly prevented diarrhea development in mice challenged with rotavirus with a no diarrhea on day 2 and a reduction of 88% on day 3 (a 52% increase in efficacy over VHH1 alone). The duration and severity of disease was also markedly reduced in the combination group, as compared to mice treated with either of the fragments (Table 1). The combination reduced diarrhea duration and severity by 64% and 84% respectively over VHH1 alone. We subsequently tested whether the combination of VHH1 and VHH3 could provide similar protection therapeutically by administering the fragments after the infection was established. When given therapeutically, VHH1 reduced diarrhea prevalence to 43% on day 2 and 28% on day 3. However, therapeutic administration of VHH3 did not protect against diarrhea. The combination of VHH1 and VHH3 was also not effective in reducing diarrhea when given therapeutically, suggesting that VHH3 may be crucial at the time of infection and therefore only works prophylactically (Table 1).

Expression of VHH1 and VHH3 in Different Combinations in lactobacillus

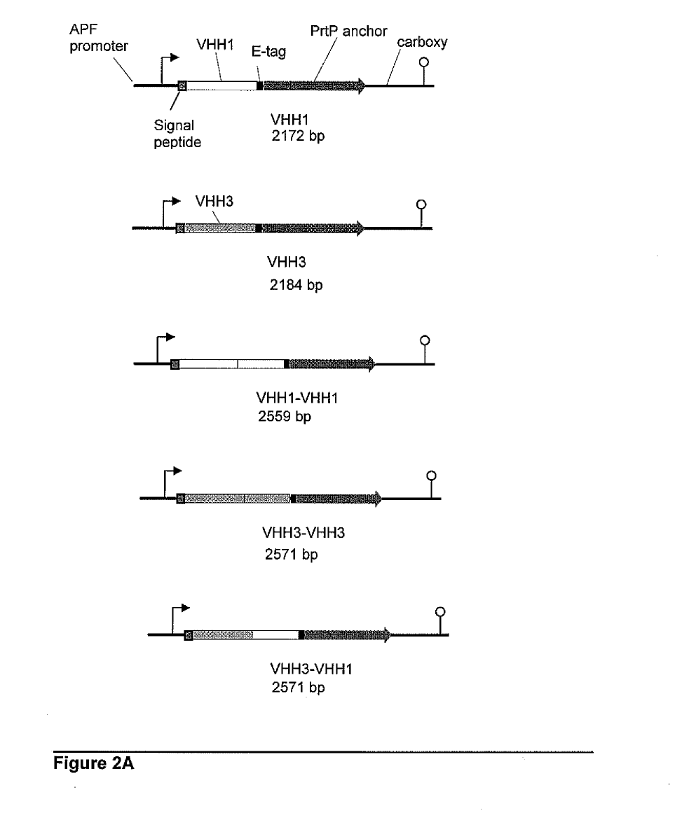

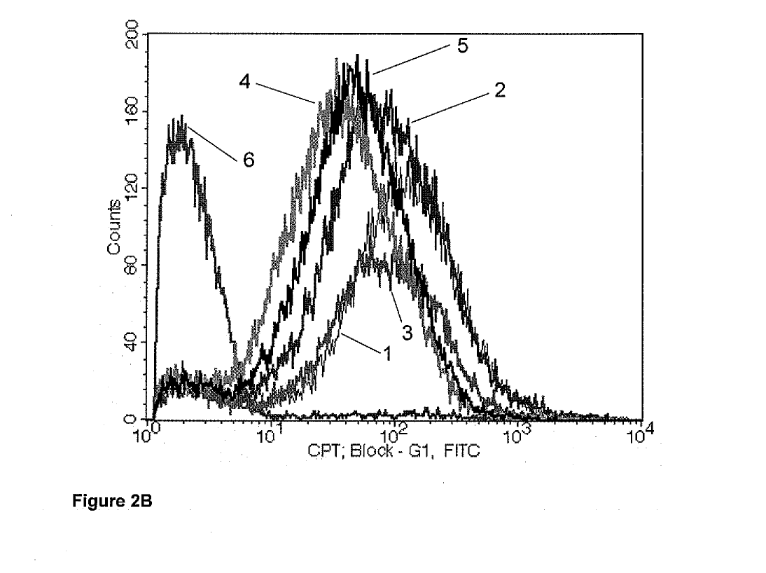

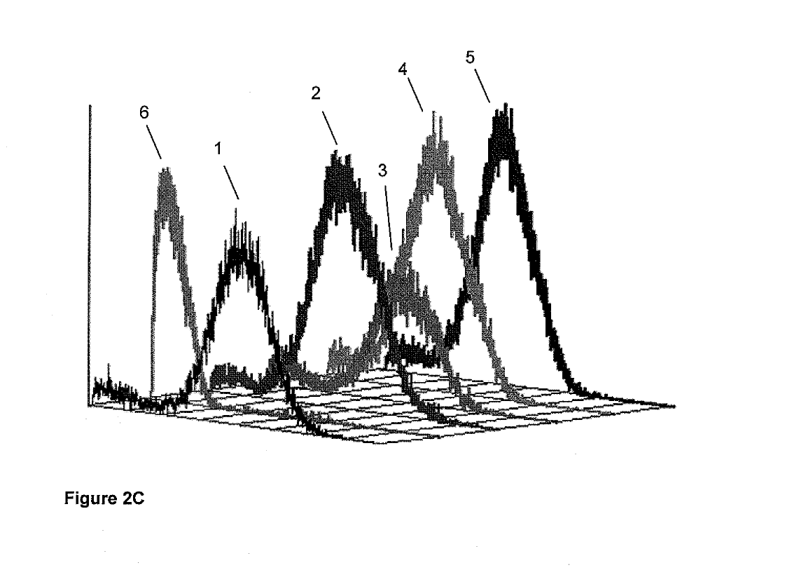

Multivalency leads to higher avidity (functional affinity) in antigen antibody interactions. We therefore expressed VHH1 and VHH3 fragments as homo- and hetero-dimers in lactobacilli (FIG. 2a). The expression of the VHH proteins on the bacterial surface was estimated by staining for the incorporated E-tag followed by flow cytometry (FIG. 2b). The results show that expression levels differ for different VHH fragments as VHH3 is expressed more efficiently than VHH1. In general, the dimers (VHH3-VHH1 and VHH3-VHH3) were expressed at comparable levels to VHH3 monomer. However, VHH1 and VHH1-VHH1 were expressed at lower levels with the latter being the least expressed. These results suggest that the expression level is not dependent on valency (as a majority of dimers were efficiently expressed) but may depend on other characteristics of the VHH fragments such as mRNA and polypeptide stability. In the beginning, to generate a dimer containing VHH1 and VHH3, we fused the domains in the reverse orientation to what is presented in this article, i.e. VHH1-VHH3. This protein was expressed very poorly in lactobacilli. However, swapping the domains to VHH3-VHH1 resolved this problem and the dimer was expressed efficiently (data not shown).

### Avidity of VHH Expressing Lactobacilli to Bind Rotavirus

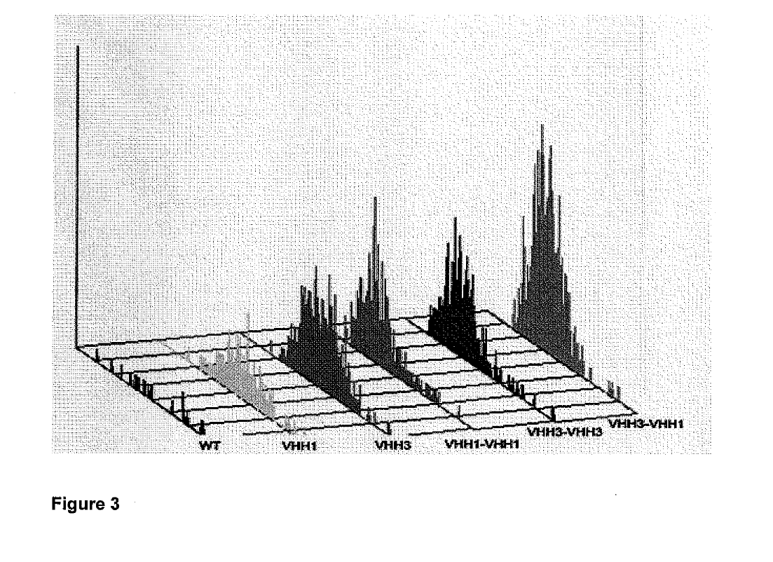

To ascertain whether the VHH fragments produced by lactobacilli were functional, we incubated the transformed lactobacilli with RRV and subsequently with rabbit anti-sera against rotavirus and anti-rabbit PE conjugated antibodies. All the lactobacilli expressing monomeric or dimeric VHHs against rotavirus bound the virus as detected by flow cytometry. VHH3 expressing lactobacilli bound more rotavirus than VHH1 expressing lactobacilli which could be due to superior expression of VHH3 as compared to VHH1. Lactobacilli expressing VHH1-VHH1 had approximately 1.5 fold improved binding to rotavirus compared to VHH1 expressing lactobacilli, even though VHH1-VHH1 was not as efficiently expressed as monomeric VHH1. This suggests that most of the bivalent VHH1-VHH1 fragments were expressed in a correct conformation to bind rotavirus and that there was a functional gain of affinity with bivalency. Although both VHH3-VHH3 and VHH3-VHH1 had the same level of expression in lactobacilli, as seen by staining for the E-tag, the latter showed a much stronger binding to rotavirus and among all the constructs tested, it was the best binder (FIG. 3).

### Fluorescence Microscopy

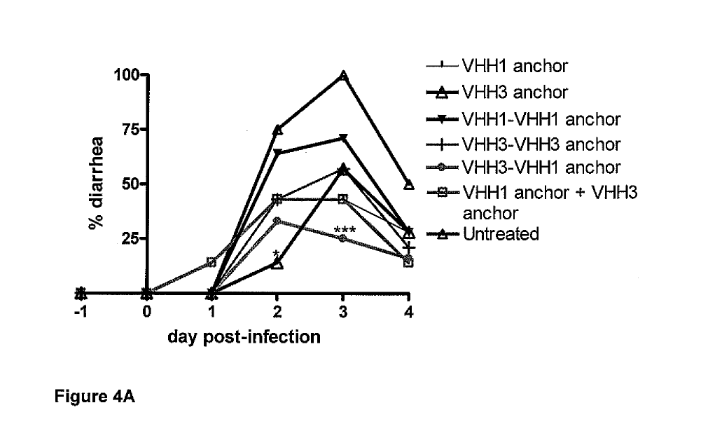

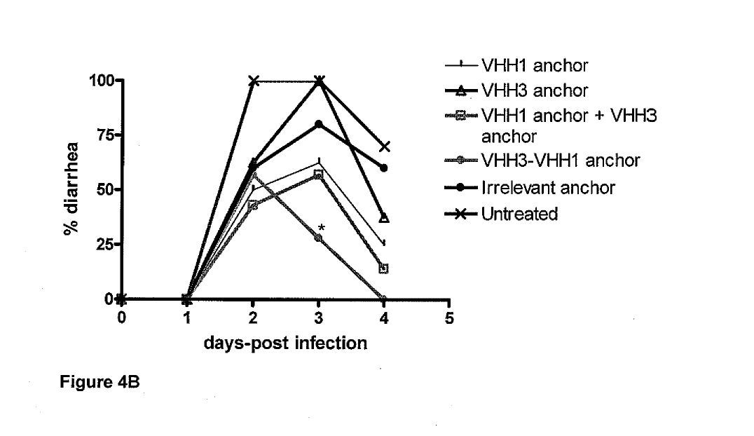

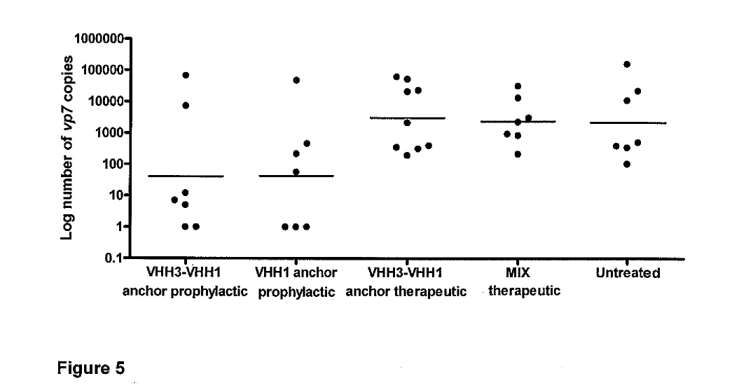

To ascertain whether VHH expressing lactobacilli could prevent the infection of MA104 cells by aggregating rotavirus we incubated lactobacilli expressing anchored VHH3-VHH1 fusion protein with rotavirus and used it for infection of MA104 cells. The cells were subsequently stained for rotavirus VP6 protein indicative of ongoing infection. Cells treated with wild type L. paracasei were marginally protected from infection with rotavirus. In comparison, cells treated with lactobacilli expressing anchored VHH3-VHH1 protein had a reduced rate of infection. Lactobacilli expressing VHH3-VHH1 bound rotavirus virions (as judged by positive staining for rotavirus VP6) and thereby helped in reducing the burden of virus particles capable of initiating independent infectious events. Wild type L. paracasei did not bind rotavirus (FIG. 4).

### In-Vivo Test/Prophylactic

Lactobacilli expressing the VHH fragments were administered to mice prophylactically, one day before infection, and the treatment was continued once daily. Treatment with VHH1 or VHH3 monomer expressing bacteria alleviated diarrhea with a significant reduction in disease prevalence and severity. Lactobacilli expressing VHH3-VHH3 dimers also reduced diarrhea, although the curative effect was not superior to that of the VHH3 monomer expressing lactobacilli. Lactobacilli expressing VHH1-VHH1 protected poorly against diarrhea. In comparison, the VHH3-VHH1 dimer expressing lactobacilli strongly reduced diarrhea prevalence on day 2 to 33% and on day 3 to 25% (a 56% and 75% improvement over infection only mice) and significantly reduced disease duration and severity (FIG. 4a and Table 2). In comparison to the VHH1 expressing lactobacilli, the lactobacilli expressing VHH3-VHH1 dimer reduced diarrhea duration by 34%.

### In-Vivo Test/Therapeutic

As therapeutic intervention, treatment with Lactobacilli was started 2 hrs after infection and continued once daily. Treatment with lactobacilli expressing monomer of VHH1 was able to reduce diarrhea severity significantly. However, as had also been observed for the yeast purified VHH3 product, VHH3 monomer expressing lactobacilli could not reduce diarrhea symptoms. Administration of a mix of VHH1 and VHH3 monomer expressing lactobacilli reduced diarrhea duration and severity and was better than the VHH1 monomer only receiving group. Treatment with lactobacilli expressing dimer of VHH3-VHH1 reduced diarrhea prevalence significantly on day 3 to 28% (a 72% improvement over infection only mice) and also reduced diarrhea duration and severity drastically (FIG. 4b and Table 2).

TABLE 2

| | | | |
|---|---|---|---|
| | Highest % | | |
| n | Prevalence | Duration ± SE | Severity ± SE |

Details of diarrheal disease developed under prophylactic
administration of VHH expressing lactobacilli.

| | | | | |
|---|---|---|---|---|
| Prophylactic | | | | |
| Treatment | | | | |
| VHH1 anchor | 7 | 43** | 1.14 ± 0.34 | 1.28 ± 0.42* |
| VHH3 anchor | 7 | 57* | 1.00 ± 0.38 | 1.14 ± 0.46* |
| VHH1-VHH1 anchor | 14 | 71 | 1.64 ± 0.22 | 2.28 ± 0.34 |
| VHH3-VHH3 anchor | 14 | 57* | 1.21 ± 0.26 | 1.57 ± 0.31* |
| VHH3-VHH1 anchor | 12 | 33 | 0.75 ± 0.22** | 1.17 ± 0.32** |
| Untreated | 12 | 100 | 2.25 ± 0.13 | 3.42 ± 0.28 |

Details of diarrheal disease developed under therapeutic
administration of VHH expressing lactobacilli.

| | | | | |
|---|---|---|---|---|
| Therapeutic Treatment | | | | |
| VHH1 anchor | 8 | 62 | 1.37 ± 0.32 | 1.75 ± 0.45* |
| VHH3 anchor | 8 | 100 | 2.00 ± 0.19 | 2.62 ± 0.26 |
| VHH3-VHH1 anchor | 7 | 57 | 0.85 ± 0.26** | 1.00 ± 0.31*** |
| VHH1 anchor + | 7 | 57 | 1.14 ± 0.34* | 1.28 ± 0.36** |
| VHH3 anchor | | | | |
| Irrelevant anchor | 5 | 80 | 2.00 ± 0.54 | 3.00 ± .89 |
| Untreated | 7 | 100 | 2.71 ± 0.18 | 4.28 ± 0.18 |

Similarly experiments can be performed using different VHH fragments in combinations that bind to the same epitopes as VHH1 and VHH3.

### Virus Load

Virus load in the intestinal sections was quantified using real time PCR against the rotavirus vp7 gene product. Mice treated prophylactically with lactobacilli expressing VHH1 monomer or dimers of VHH3-VHH1 had reduced virus load as compared to the untreated mice. However mice that received therapeutic treatment with VHH3-VHH1 expressing lactobacilli did not have a reduction in the viral load compared to the untreated group (FIG. 6).

### REFERENCES

1. Parashar, U. D., et al., Global illness and deaths caused by rotavirus disease in children. Emerg Infect Dis, 2003. 9(5): p. 565-72.
2. Martinelli, D., et al., Large outbreak of viral gastroenteritis caused by contaminated drinking water in Apulia, Italy, May-October 2006. Euro Surveill, 2007. 12(4): p. E070419 1.
3. Pant, N., et al., Effective prophylaxis against rotavirus diarrhea using a combination of Lactobacillus rhamnosus GG and antibodies. BMC Microbiol, 2007. 7(1): p. 86.
4. Sarker, S. A., et al., Successful treatment of rotavirus diarrhea in children with immunoglobulin from immunized bovine colostrum. Pediatr Infect Dis J, 1998. 17(12): p. 1149-54.
5. Hamers-Casterman, C., et al., Naturally occurring antibodies devoid of light chains. Nature, 1993. 363(6428): p. 446-8.
6. Frenken, L. G., et al., Isolation of antigen specific llama VHH antibody fragments and their high level secretion by Saccharomyces cerevisiae. J Biotechnol, 2000. 78(1): p. 11-21.
7. Allen, S. J., et al., Probiotics for treating infectious diarrhea. Cochrane Database Syst Rev, 2004(2): p. CD003048.
8. Pant, N., et al., Lactobacilli expressing variable domain of llama heavy-chain antibody fragments (lactobodies) confer protection against rotavirus-induced diarrhea. J Infect Dis, 2006. 194(11): p. 1580-8.
9. Coppieters, K., et al., Formatted anti-tumor necrosis factor alpha VHH proteins derived from camelids show superior potency and targeting to inflamed joints in a murine model of collagen-induced arthritis. Arthritis Rheum, 2006. 54(6): p. 1856-66.
10. van der Vaart, J. M., et al., Reduction in morbidity of rotavirus induced diarrhea in mice by yeast produced monovalent llama-derived antibody fragments. Vaccine, 2006. 24(19): p. 4130-7.
11. Acedo-Felix, E. and G. Perez-Martinez, Significant differences between Lactobacillus casei subsp. casei ATCC 393T and a commonly used plasmid-cured derivative revealed by a polyphasic study. Int J Syst Evol Microbiol, 2003. 53(Pt 1): p. 67-75.
12. Overbergh, L., et al., Validation of real-time RT-PCR assays for mRNA quantification in baboons. Cytokine, 2005. 31(6): p. 454-8.
13. Holliger, P. and P. J. Hudson, Engineered antibody fragments and the rise of single domains. Nat Biotechnol, 2005. 23(9): p. 1126-36.
14. Stijlemans, B., et al., Efficient targeting of conserved cryptic epitopes of infectious agents by single domain antibodies. African trypanosomes as paradigm. J Biol Chem, 2004. 279(2): p. 1256-61.
15. De Genst, E., et al., Molecular basis for the preferential cleft recognition by dromedary heavy-chain antibodies. Proc Natl Aced Sci USA, 2006. 103(12): p. 4586-91.

### EXAMPLE 2

Compositions and preparations of ice creams containing encapsulated anti-rotavirus VHH's or a Lactobacillus producing these VHH's. The following example of an ice cream composition is a food product according to the invention;

| | weight % |
|---|---|
| Sucrose | 13.000 |
| Skimmed Milk Powder | 10.000 |
| Butter fat | 8.000 |
| Maltodextrin 40 | 4.000 |
| Monoglycerol Palmitate (MGP) | 0.300 |
| Locust Bean Gum | 0.144 |
| Carageenan L100 | 0.016 |
| Flavour | 0.012 |
| VHH's mixture (VHH1 and VHH3 at equal amounts). | |
| at a volume resulting in between 5 and 5,000 microgram | |
| of VHH per serving. | |
| Water | to 100 |

Total soluble solids; 35% by weight, Ice content at −18° C.; 54% by weight

All the ice cream ingredients are mixed together using a high shear mixer for approximately 3 minutes. The water is added at a temperature of 80° C. The temperature of the water ice mix is approximately 55-65° C. after mixing.

The mix is then homogenized (2000 psi) and passed through to a plate heat exchanger for pasteurization at 81° C. for 25 seconds. The mix is then cooled to approximately 4° C. in the plate heat exchanger prior to use.

Alternatively, an anti-rotavirus VHH's producing Lactobacillus strain can be added instead of one or both the (encapsulated) VHH fragments, preferably in a concentration of 10⁹ per serving or higher.

The ice cream pre-mix is then frozen using a Technohoy MF 75 scraped surface heat exchanger, e.g. with no overrun introduced into the ice cream. The ice cream can be extruded at a temperature of from −4.4° C. to −5.4° C. The product can then be hardened in a blast freezer at −35° C., then stored at −25° C.

A water ice solution having the following composition was prepared as follows;

| | % by weight |
|---|---|
| Sucrose | 25 |
| Locust Bean Gum | 0.5 |
| (Encapsulated) VHH's mixture (VHH1 and VHH3 at equal | |
| amounts). at a volume resulting in between 5 and 5000 | |
| microgram of VHH per serving. | |
| water | to 100 |

Total soluble solids; 25.5% by weight, Ice content at −18° C.; 62% by weight

All the water ice ingredients are mixed together using a high shear mixer for approximately 3 minutes. The water is added at a temperature of 80° C. The temperature of the water ice mix is approximately 55-65° C. after mixing.

The mix is then homogenized (2000 psi) and passed through to a plate heat exchanger for pasteurization at 81° C. for 25 seconds. The mix is then cooled to approximately 4° C. in the plate heat exchanger prior to use.

Alternatively, an anti-rotavirus VHH's producing Lactobacillus strain can be added instead of the solution comprising encapsulated VHH's, preferably in a concentration of 10⁹ per serving or higher. Instead of the encapsulated VHH solution a anti-rotavirus VHH producing Lactobacillus strain can be added preferably in a concentration of 10⁹ per serving or higher.

The water ice solution may be frozen in a Technohoy MF 75 scraped surface heat exchanger with an overrun (volume fraction of air) of 30%. The water ice may be extruded at a temperature of from −3.8° C. to −4.5° C. The product may then be hardened in a blast freezer at −35° C., and stored at −25° C.

### EXAMPLE 3

Compositions for spreads containing (encapsulated) anti-rotavirus VHH mixture (VHH1 and VHH3 at equal amounts).or one or two Lactobacillus producing one or both VHH or one fragment and one strain producing the other fragment.

Spreads were made according to standard procedure as known in the art, using the compositions as given in Table 3.

TABLE 3
Spread compositions

| | Amount | Amount | Amount | Amount |
|---|---|---|---|---|
| | (wt. %) | (wt. %) | (wt. %) | (wt. %) |
| Component | Example 1 | Example 2 | Example 3 | Example 4 |
| Fat blend | 39.71 | 39.71 | 39.71 | 39.71 |
| Bolec ZT | 0.05 | 0.05 | 0.05 | 0.05 |
| Hymono 8903 | 0.16 | 0.16 | 0.16 | 0.16 |
| β-carotene | 0.08 | 0.08 | 0.08 | 0.08 |
| (1% in Sunflower oil) | | | | |
| Total fat phase | 40.00 | 40.00 | 40.00 | 40.00 |
| Tap water | up to 60 | up to 60 | up to 60 | up to 60 |
| Sour whey powder | 0.27 | 0.27 | 0.27 | 0.27 |
| NaCl | 0.48 | 0.48 | 0.48 | 0.48 |
| K-sorbate | 0.12 | 0.12 | 0.12 | 0.12 |
| Gelatin | 1.10 | 1.10 | 1.10 | 1.10 |
| Citric acid | To pH 4.6 | To pH 5.0 | To pH 4.6 | To pH 5.0 |
| Xanthan gum | 0.10 | 0.10 | 0.10 | 0.10 |
| Calcium salt | | | | |
| TCP C13-13 | | | 1.27 | 1.27 |
| CaSO₄•0.5H₂O | 1.74 | 1.74 | | |
| pH waterphase set | 4.6 | 5.0 | 4.6 | 5.0 |
| Encapsulated VHH sol. | 5-5000 μg | 5-5000 μg | 5-5000 μg | 5-5000 μg |
| Probiotic bacteria*¹ | 10⁶-10¹¹ per | 10⁶-10¹¹ per | 10⁶-10¹¹ per | 10⁶-10¹¹ per |
| | 100 g | 100 g | 100 g | 100 g |
| Total water phase | To 100 | To 100 | To 100 | To 100 |
| Total | 100.00 | 100.00 | 100.00 | 100.00 |

Instead of the (encapsulated) VHH solution after the last heating step also an anti-rotavirus VHH producing Lactobacillus strain can be added aseptically, preferably in a concentration of 10⁹ per serving or higher.

In the sequence listing below, SEQ ID #1 corresponds to VHH1, etc.

1-26. (canceled)

27. A food product or pharmaceutical preparation comprising a synergistic combination of at least two different antibodies or antibody fragments which are directed against a virus, wherein the antibodies are heavy chain immunoglobulins or fragments thereof of the VHH or VNAR type, or domain antibodies (dAbs) of the heavy or light chains of immunoglobulins or fragments thereof.

28. A food product or pharmaceutical preparation according to claim 27, wherein the virus is an enteropathogenic rotavirus.

29. A food product or pharmaceutical preparation according to claim 27, comprising antibodies or antibody fragments having at least 80% amino acid sequence identity with SEQ ID NO: 1 or SEQ ID NO: 3.

30. A food product or pharmaceutical preparation according to claim 27, wherein the antibodies or antibody fragments specifically bind to the same epitopes as SEQ ID NO: 1 or SEQ ID NO: 3.

31. A food product or pharmaceutical preparation according to claim 27, comprising a heterodimer of SEQ ID NO: 3 and SEQ ID NO: 1.

32. A food product or pharmaceutical preparation according to claim 27, comprising a microorganism transformed to express the antibodies or antibody fragments of claim 1.

33. A food product or pharmaceutical preparation according to claim 32, wherein the antibodies or antibody fragments are expressed and/or secreted in the gut by a probiotic microorganism.

34. A food product or pharmaceutical preparation according to claim 32, wherein the antibody or antibody fragments are expressed as a heterodimer of SEQ ID NO: 3 and SEQ ID NO: 1.

35. A food product or pharmaceutical preparation according to claim 27, wherein the antibodies are heavy chain immunoglobulins or fragments thereof of the VHH or VNAR type and are derived from Camelids.

36. A food product or pharmaceutical preparation according to claim 35, wherein the Camelid antibodies are llama heavy chain antibodies or fragments thereof.

37. A method for making a food product or pharmaceutical preparation according to claim 27, comprising adding the antibodies or antibody fragments during the manufacture of the food product or pharmaceutical preparation or an ingredient thereof.

38. A food product or pharmaceutical preparation produced according to the method of claim 37.

39. A method of inhibiting an enteropathogenic microoraganism infection in a subject in need thereof comprising administering an amount of the food product or pharmaceutical preparation of claim 27 effective to inhibit growth of the microorganism.

40. The method of claim 39, wherein the enteropathogenic microorganism is a virus.

41. The method of claim 40, wherein the virus is a rotavirus.

42. A food product or pharmaceutical preparation according to claim 39, wherein administration of the food product or pharmaceutical composition has an anti-diarrheal effect.

43. A food product dispensing implement comprising a probiotic microorganism, wherein the dispensing implement is coated on at least one surface with heavy chain immunoglobulins or fragments thereof of the VHH or VNAR type, or domain antibodies (dAbs) of the heavy or light chains of immunoglobulins or fragments thereof, which are active in the gut.
